(19) United States
(12) Patent Application Publication
(10) Pub. No.: US 2022/0096249 A1
(43) Pub. Date: Mar. 31, 2022

(54) CONTROL USING AN UNCERTAINTY METRIC

(71) Applicant: X Development LLC, Mountain View, CA (US)

(72) Inventors: Elliott J. Rouse, Ann Arbor, MI (US); Georgios Evangelopoulos, Venice, CA (US); Kathryn Jane Zealand, Palo Alto, CA (US)

(21) Appl. No.: 17/390,339

(22) Filed: Jul. 30, 2021

(30) Foreign Application Priority Data
Sep. 25, 2020 (GR) 20200100583

(51) Int. Cl. A61F 2/72; A61B 5/00; A61B 5/11; G06F 3/01

(52) U.S. Cl. CPC A61F 2/72; A61B 5/7289; A61B 5/7282; A61B 5/112; A61F 2002/6827; A61B 5/7264; G06F 3/011; G06F 3/016; A61B 5/7221

(57) ABSTRACT

Methods, systems, and apparatus, including computer programs encoded on a computer storage medium, for an exosuit activity transition control structure. In some implementations, sensor data representing an estimate of sensor data that will be produced by sensors of an exosuit at a particular time in the future is generated, where the exosuit is configured to assist mobility of a wearer. Actual sensor data that is generated using the sensors of the exosuit is obtained. A measure of uncertainty is determined based on the forecasted sensor data and the actual sensor data. Based on the measure of uncertainty, a control action for the exosuit to adjust an assistance provided to the wearer is determined.

### CROSS-REFERENCE TO RELATED APPLICATION

This application claims priority to Greece Application 20200100583 filed on Sep. 25, 2020. The disclosure of this prior application is considered part of and is incorporated by reference in its entirety in the disclosure of this application.

### FIELD

This disclosure generally relates to electronic control systems.

### BACKGROUND

Control systems can be used to control various devices, such as autonomous or semi-autonomous vehicles as well as exosuits.

### SUMMARY

In some implementations, a control system can determine an uncertainty metric that indicates a level of uncertainty regarding a current situation or mode of control of a device. The control system can use the uncertainty metric to change how the device is controlled. For example, the control system for a device can include hardware and software components that determine an uncertainty score, and the control system can perform actions based on the determined uncertainty score. To determine a level of uncertainty, the control system can predict values of sensor measurements that are expected to be received at a time in the future (e.g., at the next 100 ms, the next second, etc.). In other words, the system can predict which values will measured by sensors in the future, by extrapolating or projecting from past sensor measurements, where the predicted sensor data would be consistent with the current motion, operation, or situation of a device. The control system can then compare the predicted sensor data with later-received actual sensor data measured, to determine how well the prediction aligns with the actual conditions that occurred. If the difference between predicted sensor data and actual sensor data is high, the control system determined that there is significant uncertainty and may modify its control instructions in response.

The control system may apply to a situation when there is a human operator and a machine, and the human operator and the machine must be in sync with one another. That is, the control system may provide a way of predicting the intent or movement of a human operator, such as a predicted pattern of movement. Specifically, the control system may be applicable to various devices that have a human operator, such as, for example, exosuits and autonomous or semi-autonomous vehicles. The uncertainty score can be based on sensor data predicted by the control system (e.g., estimated readings of one or more sensors at a particular point in time) and sensor data collected by the control system from the one or more sensors of the device and, and may indicate a degree of disparity between the predicted sensor data and the collected sensor data. The control system can use the uncertainty score to select an action to perform or select a particular class of action to perform. Potential actions to perform may be predetermined and may be associated with a particular uncertainty score value, or a particular range of uncertainty score values. Particular classes of actions may include a class for adjusting motion or movement of the device, prompting a user of the device for user input, adjusting thresholds and/or algorithms for selecting an action or a class of actions, etc. As an example, the selectable actions for an action class of adjusting motion or movement of the device may include modifying a control settings such as reducing the amount of assistance provided by a powered exosuit at a joint of the powered exosuit, selecting a control program to run such as a safety program to reduce the risk of injury to a wearer of a powered exosuit, or modifying a threshold to perform a particular action.

The control system can use previously collected sensor data to predict sensor data that the one or more sensors are expected to produce at a future point in time, such as at a predetermined time in the future. Later, the control system can collect sensor data measured at the future point in time using the one or more sensors of the device. The control system uses the predicted sensor data and the collected sensor data to determine the uncertainty score. For example, the uncertainty score may be a vector distance between (i) a vector representing the collected sensor data (e.g., the actual sensor data) for a particular time and (ii) a vector representing the predicted sensor data that was predicted to occur at the particular time.

In some implementations, the control system can use one or more machine learning models. As an example, the control system may use a machine learning model to determine the uncertainty score. The machine learning model for determining the uncertainty score may receive collected sensor data including, for example, most recently collected sensor data and/or previously collected sensor data as input. The machine learning model may additionally receive the most recently determined uncertainty score as input. The machine learning model may be trained using previously collected sensor data, previously predicted sensor data, and expected uncertainty scores for the previously collected sensor data and corresponding predicted sensor data. The machine learning model may be trained with the objective of generating an output that matches the expected uncertainty scores. The output of the machine learning model may be a new uncertainty score.

The control system may additionally or alternatively use a machine learning model for predicting sensor data for some future point in time based on the collected sensor data. Similarly, the control system may additionally or alternatively use a machine learning model to determine a control action to perform based on the determined uncertainty score.

In some implementations, the control system can perform one or more actions in response to the determined uncertainty score. For example, where the device is a powered exosuit, the control system select an action to modify a control setting of the powered exosuit, such as a setting that controls the level of assistance provided by the powered exosuit. The control system may modify the settings to reduce the level of assistance provided by the powered exosuit when the uncertainty score meets or exceeds a particular threshold. As another example, the control system can change or select a control program such as a safety program for the powered exosuit in order to improve the stability provided to the wearer in order to reduce the risk of injury.

In some implementations, the control system selects multiple control actions to perform. As an example, a determined uncertainty score may meet or exceed multiple thresholds corresponding to multiple control actions. In response, the control system may select those multiple corresponding control actions to perform.

In some implementations, the control system uses collected sensor data to select a control action for the device. However, the control system may modify the way it controls the device based on the uncertainty score, for example, to override the selected control action with an alternative control action or to modify the selected control action. For example, where the device is a powered exosuit, the control system may determine, based on the collected sensor data, that the wearer is likely walking up stairs. The control system may use the collected sensor data along with corresponding predicted sensor data to determine an uncertainty score. The control system may input the uncertainty score to an algorithm that is used to modify the selected control action when the control action is an adjustment to the assistive force provided by the powered exosuit. For example, based on the uncertainty score, a selected control action of increasing the assistive force provided by the powered exosuit to 70% may be reduced to 50%.

In some implementations, the control system uses the determined uncertainty score as an input to an algorithm for calculating a control action. As an example, the control system may compare the uncertainty score to a threshold to determine that a particular control action should be performed, such as modifying a flex angle range of a joint of a powered exosuit. The control system may proceed to lookup a corresponding algorithm for modifying the flex angle range of the joint of the powered exosuit that uses the most recently determined uncertainty score as an input. For example, based on an uncertainty score of 8, the control system may use an algorithm to determine that the flex angle range for the powered exosuit should be 120°-170°.

In one general aspect, a method performed by one or more processors, includes: generating, by the one or more processors, forecasted sensor data representing an estimate of sensor data that will be produced by sensors of an exosuit at a particular time in the future, the exosuit being configured to assist mobility of a wearer; obtaining, by the one or more processors, actual sensor data that is generated using the sensors of the exosuit, the actual sensor data representing a state or condition of the exosuit at the particular time; determining, by the one or more processors, a measure of uncertainty based on the forecasted sensor data and the actual sensor data; and based on the measure of uncertainty, determining, by the one or more processors, a control action for the exosuit to adjust assistance provided to the wearer.

Implementations may include one or more of the following features. For example, in some implementations, generating the forecasted sensor data includes generating the forecasted sensor data representing an estimate of sensor data that will be produced by sensors of the exosuit at the particular time in the future.

In some implementations, generating the forecasted sensor data representing an estimate of the sensor data that will be produced by the sensors of the exosuit includes generating at least one of the following: a forecasted joint angle of an angle sensor of the exosuit; a forecasted force of a force sensor of the exosuit; a forecasted pressure of a pressure sensor of the exosuit; or an acceleration of an accelerometer or an inertial measurement unit of the exosuit.

In some implementations, generating the forecasted sensor data representing an estimate of the sensor data that will be produced by the sensors of the exosuit includes generating forecasted sensor data repeatedly for each of multiple successive time periods; and determining a measure of uncertainty based on the forecasted sensor data and the actual sensor data includes determining multiple measures of uncertainty each corresponding to one of the successive time periods.

In some implementations, generating forecasted sensor data repeatedly includes: generating forecasted sensor data periodically; or generating forecasted sensor data in response to detecting an event.

In some implementations, the sensors comprise one or more of: a force sensor; a pressure sensor; an accelerometer; a position sensor; an angle sensor; a photoelectric sensor; a time-of-flight sensor; and an inertial measurement unit.

In some implementations, determining the measure of uncertainty includes determining a measure of similarity between the forecasted sensor data and the actual sensor data.

In some implementations, determining the measure of similarity between the forecasted sensor data and the actual sensor data includes comparing the forecasted sensor data with the actual sensor data.

In some implementations, determining the measure of uncertainty based on the forecasted sensor data and the actual sensor data includes determining a distance metric indicating a level of difference between the forecasted sensor data and the actual sensor data.

In some implementations, determining the measure of uncertainty based on the forecasted sensor data and the actual sensor data includes determining a vector distance between a first vector corresponding to the forecasted sensor data and a second vector corresponding to the actual sensor data, where the first vector and the second vector each include values corresponding to multiple different sensors.

In some implementations, determining the measure of uncertainty based on the forecasted sensor data and the actual sensor data includes: providing the forecasted sensor data and the actual sensor data to a machine learning model; and obtaining the measure of uncertainty as an output of the machine learning model.

In some implementations, determining the measure of uncertainty based on the forecasted sensor data and the actual sensor data includes determining the measure of uncertainty based on one or more of: multiple forecasts of sensor data corresponding to different time periods; or multiple measurements of sensor data corresponding to different time periods.

In some implementations, determining the measure of uncertainty based on the forecasted sensor data and the actual sensor data includes determining the measure of uncertainty based on a comparison of outputs of multiple sensors of the sensors.

In some implementations, determining the control action for the exosuit based on the measure of uncertainty includes determining, based on the measure of uncertainty, to perform at least one of: modifying control settings of the exosuit; modifying a threshold for taking a particular control action; activating one or more safety rules; or selecting a control program to run.

In some implementations, determining the control action includes modifying the control settings for the exosuit, and determining to modify a control setting of the exosuit includes determining to modify at least one of: a level of power assistance provided by the exosuit or a portion of the exosuit; a maximum flex angle of a joint of the exosuit; a minimum flex angle of a joint of the exosuit; a joint angle of a joint of the exosuit; or a maximum flex angle speed at a joint of the exosuit.

In some implementations, determining the control action for the exosuit based on the measure of uncertainty includes: comparing the measure of uncertainty to a threshold measure of uncertainty; and selecting a control action to perform based on the measure of uncertainty meeting the threshold measure of uncertainty.

In some implementations, the method includes: generating instructions to perform the determined control action; and sending the instructions to one or more control devices.

In some implementations, sending the instruction to the one or more control devices includes sending the instructions to one or more of the following: an actuator configured to provide rotational force at a joint of the exosuit; an actuator configured to provide linear force between two or more components of the exosuit; or a locking mechanism a joint of the exosuit.

In another general aspect, a method includes: generating, by the one or more processors, forecasted sensor data representing an estimate of sensor data that will be produced by sensors of an autonomous or semi-autonomous vehicle at a particular time in the future, the vehicle being configured to provide assistance in a person's use or operation of the vehicle; obtaining, by the one or more processors, actual sensor data that is generated using the sensors of the vehicle, the actual sensor data representing a state or condition of the vehicle at the particular time; determining, by the one or more processors, a measure of uncertainty based on the forecasted sensor data and the actual sensor data; and based on the measure of uncertainty, determining, by the one or more processors, a control action for the vehicle.

In some implementations, generating the forecasted sensor data includes generating the forecasted sensor data representing the estimate of sensor data that will be produced by sensors of the vehicle at the particular time in the future.

In some implementations, determining the control action includes modifying the control settings for the vehicle, and determining to modify a control setting of the vehicle includes determining to modify at least one of: a level of throttle of the vehicle; a level of braking of the vehicle; or a steering angle of the vehicle.

Other embodiments of these aspects include corresponding systems, apparatus, and computer programs encoded on computer storage devices, configured to perform the actions of the methods. A system of one or more computers can be so configured by virtue of software, firmware, hardware, or a combination of them installed on the system that, in operation, cause the system to perform the actions. One or more computer programs can be so configured by virtue having instructions that, when executed by data processing apparatus, cause the apparatus to perform the actions.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 illustrates a perspective diagram of a powered exosuit having multiple hardware components, and an example block diagram for determining a control action for the powered exosuit based on an uncertainty score.

FIG. 2 is a block diagram that illustrates an example control system of a powered exosuit that is used to determine an uncertainty score and one or more control actions.

FIG. 3 are example charts illustrating the use of actual sensor data to generate predicted sensor data, and the determination of an uncertainty score and control action based on the predicted sensor data and actual sensor data for various times.

FIG. 4 is an example table that illustrates various uncertainty score ranges and corresponding control actions.

FIG. 5 illustrates an example block diagram to control a vehicle based on a determined uncertainty score.

FIG. 6 is a flowchart illustrating an example process for controlling a device based on a determined uncertainty score.

Like reference numbers and designations in the various drawings indicate like elements.

### DETAILED DESCRIPTION

A control system for a device can include hardware and software components for determining an uncertainty score and performing actions based on the determined uncertainty score. The control system may be applicable to various devices, such as, for example, exosuits and autonomous or semi-autonomous vehicles. The uncertainty score can be based on sensor data predicted by the control system (e.g., estimated readings of one or more sensors at a particular point in time) and sensor data collected by the control system from the one or more sensors of the device and, and may indicate a degree of disparity between the predicted sensor data and the collected sensor data. The control system can use the uncertainty score to select an action to perform. Potential actions to perform may be predetermined and may be associated with a particular uncertainty score value, or a particular range of uncertainty score values. As an example, the selectable actions may include modifying a control settings such as reducing the amount of assistance provided by a powered exosuit at a joint of the powered exosuit, selecting a control program to run such as a safety program to reduce the risk of injury to a wearer of a powered exosuit, or modifying a threshold to perform a particular action.

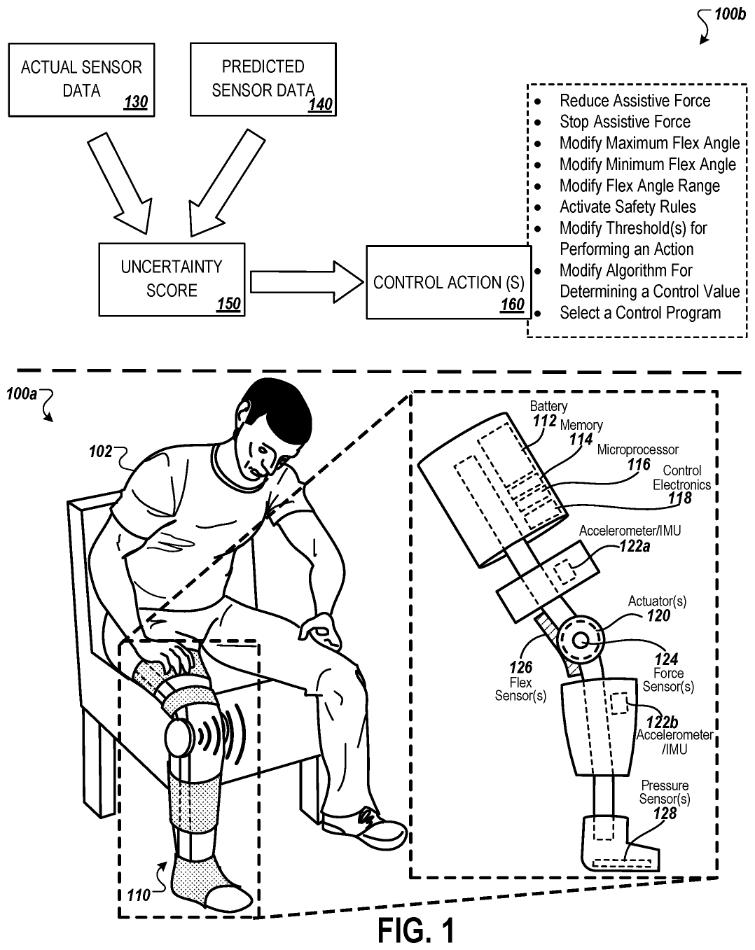

As will be described in more detail below with respect to FIG. 1, the hardware and software components of the control system can use previously collected sensor data in predicting sensor data of the one or more sensors at some future point in time. Similarly, the hardware and software components of the control system can collect sensor data at the future point in time using the one or more sensors of the device. The control system may compare the collected sensor data with the predicted sensor data to determine the uncertainty score.

The control system can be part of and/or used with a variety devices, such as powered exosuits and autonomous or semi-autonomous vehicles. For example, the control system can be used with a single leg exosuit, a lower leg exosuit, a lower body exosuit, an arm exosuit, an upper body exosuit, or the like. Similarly, the control system can be used with a vehicle (e.g., car, truck, airplane, helicopter, etc.) that uses GPS, lidar, ultrasonic sensors, cameras, altitude sensors, and/or other sensors to perform navigation functions (e.g., to perform automatic braking, lane assist or adaptive lane assist, adaptive cruise control, fully autonomous navigation, autopilot, etc.).

In general, a powered exosuit includes one or more motors or actuators that can be used to apply a force on a wearer (e.g., at a joint), to lock components of the exosuit, and/or to unlock components of the exosuit. For example, an actuator can be used to apply a torque at a knee joint of an exosuit wearer in order to assist the wearer in climbing a set of stairs.

In general, the uncertainty score determined by the control system may be a measure of confidence, such as a measure of confidence of the control system in accurately predicting the collected sensor data (e.g., a measure of confidence in the predicted sensor data). For example, the uncertainty score may be a confidence of the control system in accurately predicting sensor readings of multiple sensors of a device. The uncertainty score may be calculated using collected sensor data and previously predicted sensor data. The collected sensor data used to determine the uncertainty score may include actual sensor data corresponding to one or more times, e.g., a group of sensor readings of the device's sensors measured at a first time (e.g., substantially a point in time), or multiple groupings of sensor readings of the device's sensors measured at multiple times. Similarly, the predicted sensor data used to determine the uncertainty score may include predictions corresponding to one or more times, e.g., a group of predicted sensor readings of the device's sensors for a first time (e.g., substantially a point in time), or multiple groupings of predicted sensor readings of the device's sensors predicted at multiple times.

The uncertainty score may be calculated in various ways. As an example, the uncertainty score may be a vector distance (e.g., a Euclidean distance or a cosine distance) between a vector representing the collected sensor data (e.g., the actual sensor data) and a vector representing the predicted sensor data. The vector representing the collected sensor data may include multiple values corresponding to multiple sensors of the device. For example, the vector representing the collected sensor data may include a value for each sensor of the device. The vector representing the collected sensor data may correspond to a particular time such that the values in the vector were generated from measurements acquired from the multiple sensors at the substantially the particular time. A given value in the vector representing the collected sensor data may be a raw measurement from a corresponding sensor, a normalized value generated from a raw measurement from the sensor, or a weighted value generated by applying a weight to raw measurement from the sensor or to a normalized value generated from the raw measurement.

Similarly, the vector representing the predicted sensor data may include multiple values corresponding to multiple sensors of the device. For example, the vector representing the predicted sensor data may include a value for each sensor of the device. The vector representing predicted sensor data may correspond to a particular time such that the values in the vector are, or are calculated from, the predicted sensor outputs of the sensors of the device at the particular time (e.g., a future time with respect to the time when the vector values are calculated). A given value in the vector representing the predicted sensor data may correspond to an expected raw measurement from a corresponding sensor, a predicted normalized value, or a predicted weighted value.

As another example, the uncertainty score may be a measure of similarity between the predicted sensor data and the collected sensor. The uncertainty score may be calculated by taking the difference between the predicted sensor data and the collected sensor data, and using the difference to calculate the uncertainty score. For example, the uncertainty score may be calculated by generating a vector from the difference between a vector representing the predicted sensor data and a vector representing the collected sensor data, and taking the root mean square (RMS) value of the generated vector. Similarly, the uncertainty score may be calculated by taking the magnitude of the generated vector.

As another example, the uncertainty score may be a measure of dependence between the predicted sensor data and the collected sensor data (e.g., actual sensor data). The control system may calculate the uncertainty score by determining a measure of dependence between a vector representing the predicted sensor data and a vector representing the collected sensor data. For example, the uncertainty score may be (or may be calculated from) a determined distance correlation between a vector representing the predicted sensor data and a vector representing the collected sensor data.

In some implementations, the uncertainty score is calculated using one or more matrices. For example, the control system may calculate the uncertainty score using a matrix representing the predicted sensor data and a matrix representing the collected sensor data. Each row of the matrices may correspond to a particular time. For example, the first row of each of the matrices may correspond to the most recent

The uncertainty score may be generated using a machine learning model. As an example, a machine learning model for determining the uncertainty score may receive collected sensor data, such as most recently collected sensor data and/or previously collected sensor data, as input. The machine learning model may additionally receive one or more other inputs. For example, the machine learning model may receive the predicted sensor data, such as the most recently predicted sensor data and/or predicted sensor data corresponding to one or more previous times, as input. As another example, the machine learning model may receive previously determined uncertainty score(s), such as the most recently determined uncertainty score, as input. A machine learning model used to generate the uncertainty score may be trained using previously collected sensor data, previously predicted sensor data, and/or previously determined or verified uncertainty scores. The goal set for the machine learning model may be to accurately represent the uncertainty in the predicted sensor data with respect to the collected sensor data (e.g., for a given time period).

The uncertainty score may correspond to a particular time. The particular time may be a set time in the future (e.g., one second, fifty time steps in the future, etc.). For example, the uncertainty score may correspond to a time T₁ and may be calculated using predicted sensor data corresponding to the time T₁ (and/or predicted sensor data corresponding to one or more earlier times) and sensor data collected at substantially the time T₁ (and/or sensor data collected at one or more earlier times).

As will be discussed in more detail below, the control system can perform one or more actions in response to the determined uncertainty score. For example, where the device is a powered exosuit, the control system select an action to modify a control setting of the powered exosuit, such as a setting that controls the level of assistance provided by the powered exosuit. The control system may modify the settings to reduce the level of assistance provided by the powered exosuit when the uncertainty score meets or exceeds a particular threshold. As another example, the control system can change or select a control program such as a safety program for the powered exosuit in order to improve the stability provided to the wearer in order to reduce the risk of injury.

In some implementations, the control system selects multiple control actions to perform. As an example, a determined uncertainty score may meet or exceed multiple thresholds corresponding to multiple control actions. In response, the control system may select those corresponding multiple control actions to perform.

The techniques disclosed in this document can improve control systems for a variety of devices, particularly those requiring sensor monitoring and quick automated responses. Specifically, the techniques disclosed can help to improve the accuracy of sensor data predictions and can improve device user safety. As an example, an uncertainty score can be generated using both actual sensor data and predicted sensor data as, for example, a measure of confidence in the accuracy of the predicted sensor data. The uncertainty score can be used to both (i) improve the accuracy of the future sensor data predictions, and (ii) to select control actions that can account for or help account for the current level of uncertainty. This has the benefit of improving accuracy of future predictions while reducing the risk of injury to a user of the corresponding device, such as a wearer of a powered exosuit.

FIG. 1 illustrates a perspective diagram 100a of a powered exosuit having multiple hardware components, and an example block diagram 100b for determining a control action for the powered exosuit based on an uncertainty score.

The perspective diagram 100b depicts an example powered exosuit 110 in use. The powered exosuit 110 includes a control system for performing actions in response to sensor outputs of multiple onboard sensors, as will be described in more detail below. The powered exosuit 110 can use the sensor outputs to predict sensor outputs at a future point in time. The more accurate the prediction, the higher the confidence the control system of the powered exosuit 110 has in the actions of a wearer 102 of the powered exosuit 110 and, therefore, the higher the confidence the control system has in the control actions that it determines to be performed. However, less accurate predictions will introduce greater uncertainty that may lead the control system selecting control actions specifically in response to the level of uncertainty, e.g., in effort to reduce the likelihood of injury to the wearer 102.

The powered exosuit 110 can be, for example, a powered exoskeleton, or a mechanically assistive piece of clothing. The powered exosuit 110 may include one or more devices that can introduce a force to the wearer 102, e.g., to a particular part of the wearer 102 such as a joint of the wearer 102. For example, as shown, the powered exosuit 110 includes actuator(s) 120 that can provide a torque at the wearer 102's right knee joint.

The powered exosuit 110 can include a batter 112 such as, for example, a lithium-ion battery. The powered exosuit 110 can additionally include memory 114 such as, for example, RAM and/or long term storage. The powered exosuit 110 may also include, as discussed above, a control system. The control system may include, for example, a microprocessor 116 and control electronics 118 used to control one or more devices of the powered exosuit 110, such as the actuator(s) 120.

The powered exosuit 110 includes a number of sensors. The sensors can include force sensors, torque sensors, pressure sensors, inertial measurement units and/or accelerometers, flex sensors such as electogoniometers, or the like. Specifically, these sensors can include, for example, accelerometers/IMUs 122, force sensor(s) 124, flex sensor(s) 126, and/or a pressure sensor(s) 128. The powered exosuit 110 may include one or more additional or alternative sensors.

One or more of the sensors of the powered exosuit 110 can be calibrated based on characteristics of the wearer 102. For example, one or more of the sensors can be calibrated based on a weight of the wearer 102 and/or based on a height of the wearer 102.

As an example, as shown, the sensor data generated by the sensors (or from the output of the sensors) can include a right knee joint angle of the wearer 102, e.g., the angle between the wearer 102's right upper leg and right lower leg. The sensor data may also includes a force currently applied to the right knee joint of the wearer 102 (e.g., that is or is based on the output of the force sensor(s) 124), a pressure on the wearer 102's right foot (e.g., that is or is based on the output of the pressure sensor(s) 128), an acceleration of the wearer 102's upper leg (e.g., that is or is based on the output off the accelerometer/IMU 122a), and an acceleration of the wearer 102's lower leg (e.g., that is or is based on the output off the accelerometer/IMU 122b).

In some implementations, the sensor data includes data outputted by one or more algorithms. For example, the force applied to the wearer 102's knee may be calculated by the control system of the powered exosuit 110 applying an algorithm to the output of the force sensor(s) 124. The algorithm can output an estimated force on the wearer 102's knee joint based on the sensor's force or torque output. The algorithm can also take into consideration other senor outputs and/or known characteristics of the wearer 102, such as a weight of the wearer 102 and/or a height of the wearer 102. Where there are multiple sensors measuring the same data, the exosuit 110 can take the average of the multiple sensor outputs. For example, there can be a right-side force sensor integrated into a right hinge of the powered exosuit 110 and a left-side force sensor integrated into a left hinge of the powered exosuit 110.

In some implementations, where the control system is part of a device other than the powered exosuit, the sensors of the device can include one or more of cameras such as visible-light cameras or infrared cameras, ultrasonic sensors, photoelectric laser sensors, position sensors (e.g., for a pedal position of a vehicle), altitude sensors, etc.

The output of the sensors (e.g., sensor data) can be collected by or provided to a microprocessor 116 of the powered exosuit 110. The microprocessor 116 can be part of the control system along with the control electronics 118. In some cases, the microprocessor 116 can be part of the control electronics 118. As will be described in more detail below, the microprocessor 116 can use the collected sensor data to generate instructions to provide to the control electronics 118. The control electronics 118 can use the instructions to perform one or more control actions, such as, for example, to provide a particular counter clockwise torque at the knee joint, to provide a particular clockwise torque at the knee joint, to prevent the knee joint from bending below a particular angle, to prevent the knee joint from extending beyond a particular angle, to introduce a series of particular torques at the knee joint, to lock the knee joint at a particular angle, etc.

In the example block diagram 100b for determining a control action for the powered exosuit 110 based on an uncertainty score, actual sensor data 130 and predicted sensor data 140 are used to generate an uncertainty score 150.

The actual sensor data 130 can include outputs of multiple sensors of the powered exosuit 110, or can be based on the outputs of multiple sensors of the powered exosuit 110. The actual sensor data 130 may correspond to a particular time, e.g., a time or substantially the time when the outputs of the sensors of the powered exosuit 110 are generate and/or collected. The particular time may be a scheduled time, and/or may correspond to a particular clock cycle (e.g., the microprocessor 116 may collect sensor data every 200 ms, every hundred million clock cycles, two times a second, etc.). The actual sensor data 130 can include multiple values corresponding to multiple sensors of the powered exosuit 110. For example, the actual sensor data 130 may include a value for each sensor of the powered exosuit 110.

As an example, the actual sensor data 130 can include the raw measurements collected from corresponding sensors, normalized values generated from the raw measurements, or weighted values generated by applying one or more weights to the raw measurements. Specifically, the microprocessor 116 may collect the outputs of the sensors (e.g., the raw measurements) of the powered exosuit 110, may normalize the outputs, and may apply multiple weights to the normalized outputs depending on the sensor that produced the corresponding output. For example, a first weight may be applied to the flex sensor(s) 126 and a second, higher weight may be applied to the force sensor(s) 124. This may indicate that the measured force at the wearer 102's right knee joint is more critical to determining the uncertainty score 150 than the measured angle of the wearer 102's right knee joint, e.g., due to a determination that accurately predicting the force at the wearer 102's right knee joint is critical to preventing injury.

The predicted sensor data 140 can include predicted outputs of multiple sensors of the powered exosuit 110, or can be based on the predicated outputs of multiple sensors of the powered exosuit 110. The predicted sensor data 140 may correspond to a particular time, e.g., a time or substantially the time when the outputs of the sensors of the powered exosuit 110 are generate and/or collected for the actual sensor data 130. That is, for example, the time corresponding to the predicted sensor data 140 and the actual sensor data 130 may be the same such that the predicted sensor data 140 is a previously generated prediction of the actual sensor data 130. Like the actual sensor data 130, the predicted sensor data 140 can include multiple values corresponding to multiple sensors of the powered exosuit 110. For example, the predicted sensor data 140 may include a value for each sensor of the powered exosuit 110.

As an example, the predicted sensor data 140 may include predicted values that correspond to expected raw measurements of multiple sensors, predicated values that correspond to normalized measurements of the multiple sensors, or predicted values that correspond to weighted measurements (e.g., weights applied to raw measurements or to normalized measurements) of the multiple sensors. Specifically, the microprocessor 116 may use the previously collected sensor data to predict the sensor data to be collected at some future point in time. The microprocessor 116 may additionally use previously predicted sensor data and/or previously determined uncertainty scores to generate the predicted sensor data 140.

The actual sensor data 130 may be a vector having multiple values that correspond to multiple sensors of the powered exosuit 110 (e.g., a value may correspond to a sensor that produced the value as an output, or a value may correspond to a sensor that produced an output that was used to calculate the value). Each of the values in the vector for may correspond to a particular time. Similarly, the predicted sensor data 140 may be a vector having multiple predicted values that correspond to multiple sensors of the powered exosuit 110 (e.g., a predicted value may correspond to a sensor that is expected to produce the value as an output, or a predicted value may correspond to a sensor that is expected to produce an output that will be used to calculate the value). Each of the predicted values in the vector for may also correspond to the particular time.

In some implementations, the actual sensor data 130 is a matrix having multiple rows of values. Each row in the matrix may correspond to a particular time, e.g., a time when the sensor measurements that were used as the values or used to calculate the values in the corresponding row were collected or otherwise obtained. Similarly, the predicted sensor data 140 may be a matrix having multiple rows of values. Each row in the matrix may correspond to a particular time, e.g., a time when the sensors are expected to output the predicted values.

In some implementations, a machine learning model is used to generate the predicted sensor data 140. For example, a machine learning model for determining the predicted sensor data 140 for a future point in time may receive collected sensor data, such as the most recently collected sensor data at the time (e.g., the actual sensor data immediately preceding the actual sensor data 130) and/or previously collected sensor data, as input. The machine learning model may additionally receive one or more other inputs. For example, the machine learning model may receive previously predicted sensor data, such as one or more sets of previously predicted sensor data corresponding to one or more previous times, as input. As another example, the machine learning model may receive previously determined uncertainty score(s), such as the most recently determined uncertainty score, as input.

A machine learning model used to generate the predicted sensor data 140 may be trained using previously collected sensor data, previously predicted sensor data, and/or previously determined uncertainty scores. The machine learning model may be trained with the goal of reducing the uncertainty score (e.g., reducing the difference between the collected sensor data and the predicted sensor data for a given point in time, reducing the distance between the collected sensor data and the predicted sensor data for a given point in time, increasing the confidence in the predicted sensor data, increasing the similarity between the predicted sensor data and the collected sensor data for a given point in time, etc.). The output of the machine learning model may be predicted sensor data for some future point in time (e.g., estimated sensor output values for multiple sensors of a device for a time when the sensor outputs will be collected). For example, the output of the machine learning model may be the predicted sensor data 140.

The control system can use the actual sensor data 130 and the predicted sensor data 140 to generate the uncertainty score 150. For example, the uncertainty score 150 can be generated by the microprocessor 116 taking the Euclidean distance between a vector representing the actual sensor data 130 and a vector representing the predicted sensor data 140. The uncertainty score 150 can indicate, for example, the control system's confidence in the predicted sensor data 140, and/or confidence in the accuracy of predicted sensor data corresponding to a future point in time.

Using the uncertainty score 150, the control system (e.g., the microprocessor 116) can select one or more control actions 160 to perform. As an example, the one or more control actions 160 selected may be based on the comparing the uncertainty score 150 to one or more thresholds. For example, each of the selected control action(s) 160 may correspond to a particular uncertainty score threshold or uncertainty score threshold range. If the uncertainty score 150 meets or exceeds a threshold of a control action (or falls within a threshold range of a control action), then the control system (e.g., the microprocessor 116) can generate instructions for that control action to be performed and can send the instructions to the control electronics 118 to perform the control action.

As another example, the one or more control actions 160 selected may be based on providing the uncertainty score 150 to an algorithm, e.g., in addition to comparing the uncertainty score 150 to multiple thresholds and/or threshold ranges. For example, the control system (e.g., the microprocessor 116) may compare the uncertainty score 150 to a threshold corresponding to reducing the assistive force provided by the actuator(s) 120. In response to determining that the uncertainty score 150 meets the threshold, the control system (e.g., the microprocessor 116) can provide the uncertainty score 150 as input to an algorithm for determining how much the assistive force should be reduced by. The algorithm, in this case, might also receive the current assistive force as an input. The output of the algorithm may indicate that the assistive force of the actuator(s) 120 should be reduced by 30% (e.g., from 60% to 30% of maximum torque).

As an example, the control action(s) 160 may include one or more of reducing assistive force provided by the actuator(s) 120 of the powered exosuit 110 (e.g., reducing assistive force from 70% of maximum force to 40% of maximum force), stopping the assistive force provided by the actuator(s) 120 of the powered exosuit 110 (e.g., reducing assistive force from 70% of maximum force to 0% of maximum force), modifying the maximum flex angle of the powered exosuit 110 at the knee joint (e.g., reducing the maximum flex angle from 180° to 120°), modifying the minimum flex angle of the powered exosuit 110 at the knee joint (e.g., increasing the minimum flex angle from 20° to 60°), modifying the flex angle range of the powered exosuit 110 at the knee joint (e.g., modifying the flex angle range from 20°-180° to 60°-110°), activating other safety rules (e.g., enabling a predetermined safety rules meant to safeguard the wearer 102 such as, for example, increasing assistive force output, providing improved stability, reducing the allowable rotation at a joint, locking a joint to prevent rotation, or the like), modifying thresholds for performing an action (e.g., modifying the threshold corresponding to triggering a control action, such as reducing assistive force, to reduce uncertainty score required to trigger the control action, thereby increasing the likelihood that the control action will be performed at a future point in time), modifying an algorithm for determining a control value (e.g., adjusting a constant in an algorithm used determine the maximum flex angle when the control action of modifying the maximum flex angle or modifying the flex angle range is selected by the control system to be performed), and/or selecting a control program (e.g., selecting a safety program for the powered exosuit 110 in order to improve the stability provided to the wearer 102 in an effort to reduce the risk of injury). A control value may refer to a control setting of the powered exosuit 110, such as, for example, a joint angle or a percentage of maximum torque. The control system may select from control actions that are in addition to or in place of those control actions shown.

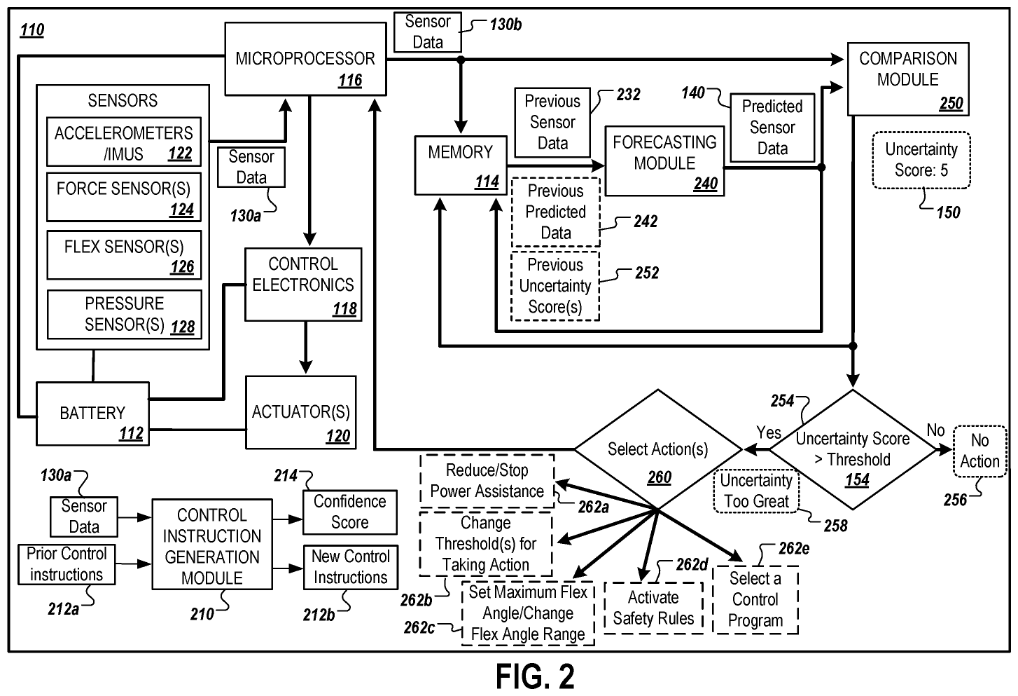

FIG. 2 is a block diagram that illustrates an example control system of the powered exosuit 110 that is used to determine an uncertainty score and one or more control actions.

As shown, the sensors of the powered exosuit 110 output sensor data 130a to the microprocessor 116 (e.g., the microprocessor 116 collects the sensor data 130a). The sensor data 130a may correspond to a particular time, e.g., a time or substantially the time when the outputs of the sensors were generated and/or measured. For example, the sensor data 130a may include the output of the accelerometers/IMUs 122 generated and/or measured at substantially a first time T₁, the output of the force sensor(s) 124 generated and/or measured at substantially the first time T₁, the output of the flex sensor(s) 126 generated and/or measured at substantially the first time T₁, and the output of the pressure sensor(s) 128 at substantially the first time T₁.

The sensor data 130a may also be provided a control instruction generation module 210. The control instruction generation module 210 may be used to generate control instructions and confidence scores corresponding to those control instructions. The control instruction generation module 210 may, in addition to the sensor data 130a, receive the prior control instructions 212a (e.g., the last output of the control instruction generation module 210) as input. In response to receiving the sensor data 130a, the control instruction generation module 210 may generate new control instructions 212b and a confidence score 214 as output. The confidence score 214 may indicate the level of confidence that the control instruction generation module 210 has in its own decision, e.g., the new control instructions 212b. That is, given the sensor data 130a and the prior control instruction 212a, the confidence score 214 may indicate how likely the new control instructions 212b are to be correct.

The microprocessor 116 may use the sensor data 130a to generate the sensor data 130b. Specifically, the microprocessor 116 may generate the sensor data 130b by normalizing the values in the sensor data 130a, and/or applying weight(s) to one or more of the values in the sensor data 130a. The microprocessor 116 may modify the sensor data 130a to generate the sensor data 130b in order to calibrate the sensor data 130a, and/or in order to account for variation between the various sensors' significance as an indicator of the uncertainty score (e.g., as a confidence indicator of the accuracy of the predicated sensor data).

The sensor data 130b may be stored in the memory 114 of the powered exosuit 110. A forecasting module 240 may access the memory 114 to retrieve previous sensor data 232 (e.g., the sensor data collected immediately preceding the sensor data 130b). The forecasting module 240 may use the previous sensor data 232 to generate the predicted sensor data 140. The forecasting module 240 may optionally use previous predicted data 242 (e.g., the most recently predicated sensor data) and/or previous uncertainty score(s) (e.g., the most recently generated uncertainty score) in generating the predicted sensor data 140. The previous sensor data 232 includes sensor data corresponding to a single previous point in time (e.g., sensor data collected at substantially one prior time), or may include sensor data corresponding to multiple previous points in time (e.g., sensor data collected over multiple prior times). Similarly, the previous predicted data 242 may include predicted sensor data that corresponds to a single previous point in time, or to multiple previous points in time. The previous uncertainty score(s) 252 may include a single uncertainty score that corresponds to a single previous point in time, or multiple uncertainty scores that each correspond to different previous points in time.

As discussed above with respect to FIG. 1, the forecasting module 240 may use a machine learning model to generate the predicted sensor data 140. The machine learning model may receive the previous sensor data 232 as input. The machine learning model may additionally receive the previous predicted data 242 and/or the previous uncertainty score(s) 252 as input. The output of the machine learning model may be a set of values, e.g., with each value corresponding to a particular sensor of the powered exosuit 110. These set of values may make up the predicted sensor data 140 or may be used by the forecasting module 240 to generate the predicated sensor data 140.

In some cases, the forecasting module 240 weighs input data that corresponds to a more recent time more heavily than input data the corresponds to a less recent time. For example, the previous sensor data 232 may include a first vector of sensor data that corresponds to a time T2 and a second vector of sensor data that corresponds to a time of T1. In generating the predicted sensor data 140, the forecasting module 240 may apply a weight of 1.2 to the first vector and a weight of 0.8 to the second vector so that the more recent sensor data is weighed more heavily when predicting sensor data for a future point in time.

The microprocessor 116 may also send the sensor data 130b to a comparison module 250. The comparison module 250 may also receive the predicted sensor data 140 from the forecasting module 240. Once the comparison module 150 has received both the sensor data 130b (e.g., the actual sensor data) and the predicted sensor data 140, the comparison module 250 may compare the sensor data 130b to the predicted sensor data 140 in order to determine the uncertainty score 150 (e.g., may compare all or part of the predicated sensor data 140 with all or part of the sensor data 130b). As an example, the comparison module 250 may determine a distance (e.g., Euclidean distance, cosine distance, etc.) between a vector representing the sensor data 130b and a vector representing the predicted sensor data 140. This distance may be provided as an input to an algorithm that is used to calculate the uncertainty score 150. In determining the uncertainty score 150, the comparison module 250 may do one or more other calculations or comparisons in addition to or in place of calculating the distance as discussed above. As an example, the comparison module 250 has calculated an uncertainty score of 0.25 based on the sensor data 130b and the predicated sensor data 140.

In some cases, the comparison module 250 generates multiple uncertainty scores. For example, the comparison module 250 may generate an uncertainty score that corresponds to each sensor of the powered exosuit 110 that data was collected from and/or predicated for. The comparison module 250 may calculate an overall uncertainty score from the multiple uncertainty scores. For example, the comparison module 250 may apply different weights to the multiple uncertainty scores and then average the multiple weighted uncertainty scores to determine an overall uncertainty score. The uncertainty score 150 may be an overall uncertainty score.

The uncertainty score 150 may be stored in the memory 114. The uncertainty score 150 may be associated with the same time as the predicated sensor data 140 and the sensor data 130b (e.g., time T₃ where the previous sensor data 232 corresponds to a time T₂).

The uncertainty score 150 may be compared to a threshold 154. If the uncertainty score does not meet or exceed the threshold 154, then a decision 256 to not perform any control actions, or any control actions based on the uncertainty score is made. However, if the uncertainty score 150 does meet or exceed the threshold 154, then a determination 258 that the uncertainty is too great is made. In response to determining that the uncertainty is too great, one or more control actions 260 are selected. As shown, selecting one or more control actions 260 can include optionally selecting a first action 262a to reduce/stop power assistance provided by the actuator(s) 120 of the powered exosuit 110, a second action 262b to change threshold(s) for taking action, a third action 262c for setting a maximum flex angle or changing a flex angle range, a fourth action 262d for activing safety rules, and/or a fifth action 262e for selecting a particular control program.

Specifically, the first action 262a provides for reducing and/or stopping the power assistance provided by the powered exosuit 110. Changing the power assistance of the powered exosuit 110 can include changing the amount of force or torque applied by the actuator(s) 120. In some cases, another control action may include increasing the amount of power assistance.

The second action 262b is changing of thresholds to take an action. For example, one or more of the control actions 262a-262e can have a corresponding threshold or a corresponding threshold range. If the second action 262b is taken, the threshold or threshold corresponding to one or more of the actions 262a-262e can be changed. For example, as a result of a significant uncertainty but perhaps not enough uncertainty to trigger stopping power assistance, the threshold for triggering the stopping power assistance may be reduced from a 8.5 to a 7.5 uncertainty score threshold.

The third action 262c provides for setting the maximum flex angle and/or changing the flex angle range for a joint of the powered exosuit 110. For example, based in the uncertainty score 150 exceeding a particular threshold, the control system may determine that the flex angle range should be modified. The control system may use an algorithm corresponding to changing the flex angle range to determine a new flex angle range. The algorithm may receive, for example, the uncertainty score 150, the current flex angle range, and/or the current flex angle of the powered exosuit 110 from the sensor data 130b as input. For example, based on the uncertainty score 150 being 5, the current flex angel range being 40°-180°, and the current angle of the knee joint of the powered exosuit 110 being 105°, the control system may use an algorithm to determine anew flex angle range of 90°-140°.

The fourth action 262d provides for the control system (e.g., the microprocessor 116) activating safety rules. The actual safety rules can be used, for example, to improve stability provided by the powered exosuit 110, to increase the amount of assistance provided by the powered exosuit 110 (e.g., by activating the actuator(s) 120 or increasing the force output of the actuator(s) 120), to decrease the amount of assistance provided by the powered exosuit 110 (e.g., by deactivating the actuator(s) 120 or decreasing the force output of the actuator(s) 120), to lock a hinge of the exosuit 110, and/or to unlock a hinge of the exosuit 110. The one or more safety rules activated can depend on the uncertainty score 150 and/or on the sensor data 130b.

The fifth action 262e provides for the control system (e.g., the microprocessor 116) selecting or changing a control program. There can be, for example, various control programs that correspond to different uncertainty scores or ranges of uncertainty scores. The control programs may additionally depend on the actual sensor data. Activating a control program for a transition can result in deactivating a previous running control program.

The control actions that are ultimately selected by the microprocessor 116 may be based on the uncertainty score 150 and/or on the sensor data 130b. For example, each of the control actions 262a-262e may have a corresponding threshold or threshold range that can be compared to the uncertainty score 150. The selected control actions may include those that correspond to a threshold that the uncertainty score 150 meets, or correspond to a threshold range that the uncertainty score 150 falls into. As another example, the control actions to be performed may be selected based on the output of a machine learning model. The machine learning model may receive the uncertainty score 150 as input. The machine learning model may additionally receive the sensor data 130b as input. The output of the machine learning model may be a value that corresponds to one or more control actions.

Upon selecting the control actions to be performed, the microprocessor 116 may generate instructions that it sends to the control electronics 118. The control electronics 118 may then perform the selected control actions using, for example, the actuator(s) 120.

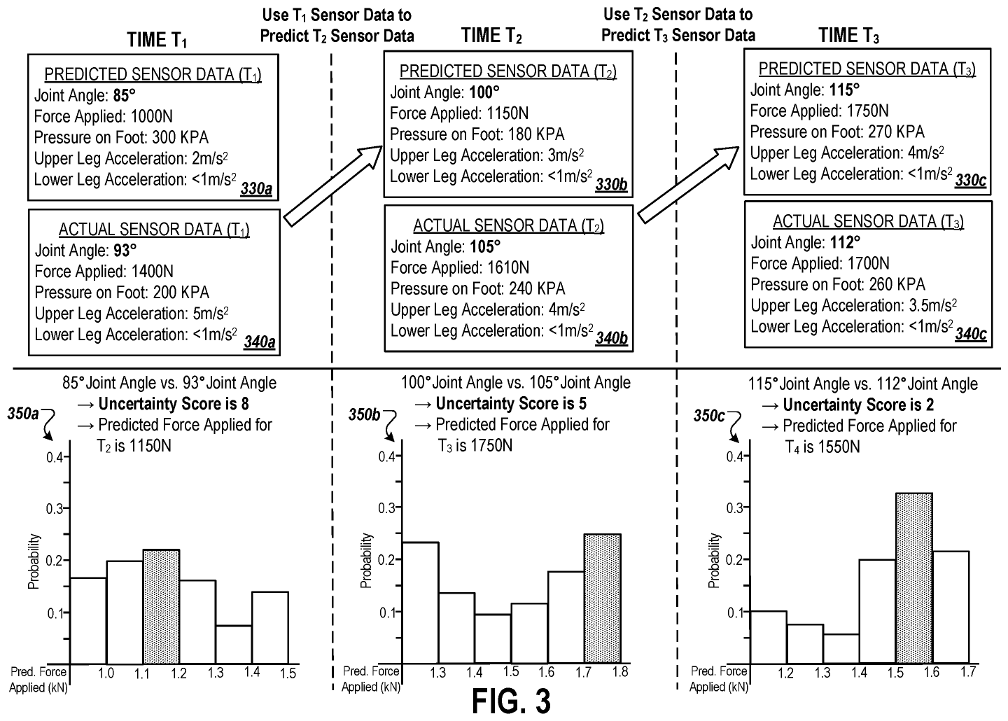

FIG. 3 are example charts illustrating the use of actual sensor data to generate predicted sensor data, and the determination of an uncertainty score and control action based on the predicted sensor data and actual sensor data for various times.

As shown, predicted sensor data 330a and actual sensor data 340a correspond to a time T₁. That is, for example, that the predicted sensor data 330a was a previously generated by the control system as a prediction of what the powered exosuit 110's sensors would read at the time T₁, and the actual sensor data 340a are the actual readings of the powered exosuit 110's sensors at the time T₁.

The control system may compare the predicated sensor data 330a with the actual sensor data 340a. For example, the control system may compare the joint angle of the predicated sensor data 330a of 85° with the joint angle of the actual sensor data 340a of 93°. Based on the comparison, the control system may determine that the current uncertainty score corresponding to the time T₁ is 8, e.g., indicating high uncertainty. Using, for example, the actual sensor data 340a and the uncertainty score of 8, the control system may refer to historical data stored in the memory 114 of the powered exosuit 110 to predict the most likely force that will be applied at the time T₂. For example, the control system may access historical data on force applied when the prior joint angle was 93° (or between 90° and 95°) and the last uncertainty score was 8. The historical data may provide, for example, that most likely force applied to be expected at the time T₂ will be between 1100N and 1200N with a probability of about 0.23. Based on this, the control system predicts that force applied for the time T₂ will be 1150N.

The control system may proceed to use other data entries in the actual sensor data 340a, differences between other data entries in the actual sensor data 340a and the predicted sensor data 330a, and/or the uncertainty score for the time T₁ (e.g., the uncertainty score of 8) to generate the rest of the predicted sensor data 330b for the time T₂.

As shown, predicted sensor data 330b and actual sensor data 340b correspond to a time T₂. That is, for example, that the predicted sensor data 330b was a previously generated by the control system as a prediction of what the powered exosuit 110's sensors would read at the time T₂, and the actual sensor data 340b are the actual readings of the powered exosuit 110's sensors at the time T₂.

The control system may compare the predicated sensor data 330b with the actual sensor data 340b. For example, the control system may compare the joint angle of the predicated sensor data 330b of 100° with the joint angle of the actual sensor data 340b of 105°. Based on the comparison, the control system may determine that the current uncertainty score corresponding to the time T₂ is 5, e.g., indicating medium uncertainty. Using, for example, the actual sensor data 340b and the uncertainty score of 5, the control system may refer to historical data stored in the memory 114 of the powered exosuit 110 to predict the most likely force that will be applied at the time T₃. For example, the control system may access historical data on force applied when the prior joint angle was 105° (or between 100° and 105°) and the last uncertainty score was 5. The historical data may provide, for example, that most likely force applied to be expected at the time T₃ will be between 1700N and 1800N with a probability of about 0.25. Based on this, the control system predicts that force applied for the time T₃ will be 1750N.

The control system may proceed to use other data entries in the actual sensor data 340b, differences between other data entries in the actual sensor data 340b and the predicted sensor data 330b, and/or the uncertainty score for the time T₂ (e.g., the uncertainty score of 5) to generate the rest of the predicted sensor data 330c for the time T₃.

As shown, predicted sensor data 330c and actual sensor data 340c correspond to a time T₃. That is, for example, that the predicted sensor data 330c was a previously generated by the control system as a prediction of what the powered exosuit 110's sensors would read at the time T₃, and the actual sensor data 340c are the actual readings of the powered exosuit 110's sensors at the time T₃.

The control system may compare the predicated sensor data 330c with the actual sensor data 340c. For example, the control system may compare the joint angle of the predicated sensor data 330c of 115° with the joint angle of the actual sensor data 340c of 112°. Based on the comparison, the control system may determine that the current uncertainty score corresponding to the time T₃ is 2, e.g., indicating low uncertainty. Using, for example, the actual sensor data 340c and the uncertainty score of 2, the control system may refer to historical data stored in the memory 114 of the powered exosuit 110 to predict the most likely force that will be applied at the time T₄. For example, the control system may access historical data on force applied when the prior joint angle was 112° (or between 110° and 115°) and the last uncertainty score was 2. The historical data may provide, for example, that most likely force applied to be expected at the time T₄ will be between 1500N and 1600N with a probability of about 0.33. Based on this, the control system predicts that force applied for the time T₃ will be 1550N.

The control system may proceed to use other data entries in the actual sensor data 340c, differences between other data entries in the actual sensor data 340c and the predicted sensor data 330c, and/or the uncertainty score for the time T₃ (e.g., the uncertainty score of 2) to generate the rest of the predicted sensor data for the time T₄.

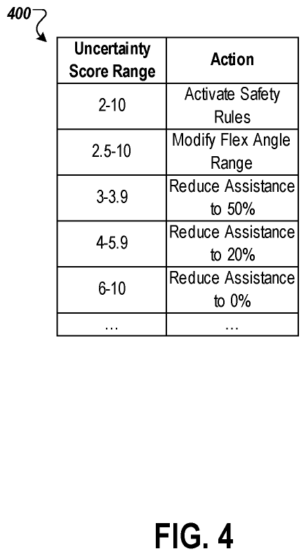

FIG. 4 is an example table 400 that illustrates various uncertainty score ranges and corresponding control actions.

The table 400 includes a first column that corresponds to uncertainty score ranges and a second column that corresponds to control actions. The control system (e.g., the microprocessor 116) may refer to the table 400 when determining what control actions should be performed based on the determined uncertainty score.

As an example, when the determined uncertainty score for a time T₅ is 3.3, the microprocessor 116 may refer to the table 400 and proceed to compare the uncertainty score with the uncertainty score ranges in the first column of the table 400. Upon determining that the uncertainty score of 3.3 falls into the uncertainty score range of 3-3.9 in the third row, the microprocessor 116 may identify the corresponding control action in the third row of the second column of the table 400 as the control action that should be performed. Specifically, using the table 400, the microprocessor 116 may select the control action of reducing the assistance to 50% of maximum torque based on the determined uncertainty score of 3.3 falling in the uncertainty score range of 3-3.9.

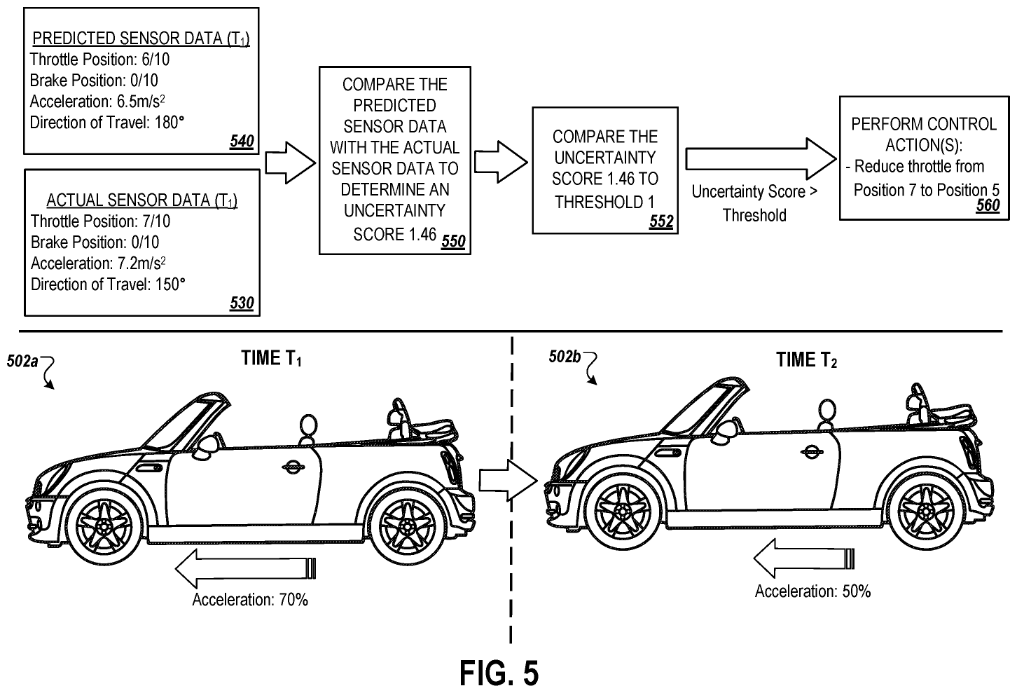

FIG. 5 illustrates an example block diagram to control a vehicle 502 based on a determined uncertainty score.

As previously mentioned, the control system described above with respect to FIGS. 1-4 is also applicable to devices other than the powered exosuit 110. Notably, the control system can be applicable to autonomous or semi-autonomous vehicles such as the vehicle 502. The sensor data collected and/or predicted for the vehicle 502 may include sensor data corresponding to differing sensors than that described above with respect to the powered exosuit 110.

As an example, the sensors for the vehicle 502 may include one or more of a position sensor for an acceleration pedal and/or a brake pedal of the vehicle 502, an accelerometer/IMU, a GPS unit, a compass, an ultrasonic sensor, a photoelectric sensor, a camera or other image sensor, etc.

At a time T₁, the vehicle 502a has an applied acceleration of 70% the vehicle 502a's maximum acceleration. The control system may have previously generated the predicted sensor data 540 for the time T₁, e.g., using actual sensor data collected at a time T₀. At the time T₁ or substantially at the time T₁, the control system may collect actual sensor data 530.

Once the control system has both the predicted sensor data 540 and the actual sensor data 530, the control system may proceed to compare the predicated sensor data with the actual sensor data to determine an uncertainty score 550. For example, the control system may generate a first vector corresponding to the predicted sensor data 540 of V₁={6, 0, 6.5, 180} and a second vector corresponding to the actual sensor data 530 of V₂={7, 0, 7.2, 150}. The control system may proceed to normalize the values in the vectors to obtain a normalized first vector of V_{1N}={0.6, 0, 0.65, 0.5} and a normalized second vector of V_{2N}={0.7, 0, 0.72, 0.42}. In order to determine the uncertainty score 550, the control system may proceed to calculate the Euclidean distance between V_{1N} and V_{2N} to obtain a distance of 0.146. The control system may multiple the distance by a constant value of, for example, ten to obtain an uncertainty score of 1.46

The control system may proceed to compare the uncertainty score 550 to a threshold 552. The control system may determine that the uncertainty score 550 meets or exceeds the threshold 552 of one, and, therefore, that a control action corresponding to the threshold 552 should be performed.

As an example, the control system may determine that a control action of reducing the throttle position by two positions should be performed based on the uncertainty score 550 exceeding the threshold 552. The control system may proceed to generate instructions to have the throttle position reduced from a current position of 7 to a position of 5, and may send the instructions to control electronics of the vehicle 502.

As a result of the control system modifying the throttle position from position 7 to position 5, the acceleration of the vehicle 502b at a time of T₂ has been reduced to 50%.

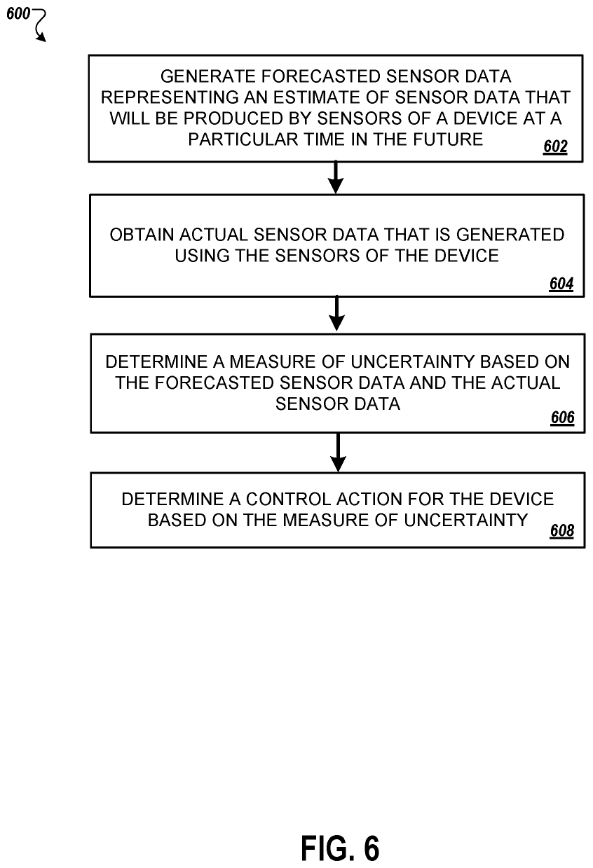

FIG. 6 is a flowchart illustrating an example process 600 for controlling a device based on a determined uncertainty score. The process 600 can be performed, at least in part, using the control system described herein.

The process 600 includes generating forecasted sensor data representing an estimate of sensor data that will be produced by sensors of a device at a particular time in the future (602). As an example, with respect to FIG. 2, the forecasting module 240 can use previously observed sensor data 232 to generate the predicated sensor data 140 for a future point in time. The forecasting module 240 may optionally use previously predicted data 242 and/or previous uncertainty score(s) 252 to generate the predicated sensor data 140. The forecasting module 240 may use an algorithm, such as a static algorithm or a machine learning algorithm to generate the predicated sensor data 140. The machine learning algorithm may be trained with the goal of reducing the uncertainty scores ultimately produced by the comparison module 250.

The process 600 includes obtaining actual sensor data that is generated using the sensors of the device (604). As an example, with respect to FIG. 2, the actual sensor data 130a may be generated by the sensors of the powered exosuit 110, such as by the accelerometers/IMUs 122, the force sensor(s) 124, the flex sensor(s) 126, and the pressure sensor(s) 128. The microprocessor 1116 may obtain the sensor data 130a from the sensors.

In some cases, the actual sensor data may be modified. For example, with respect to FIG. 2, control system (e.g., the microprocessor 116) may adjust the sensor data 130a by normalizing the values in the sensor data 130a and/or by applying weights to the values in the sensor data 130a.

The process 600 includes determining a measure of uncertainty based on the forecasted sensor data and the actual sensor data (606). The measure of uncertainty may be the uncertainty score described herein. As described above, the uncertainty score (e.g., the uncertainty measure) may be a measure of confidence in the predicted sensor data, a measure of distance or difference between the predicted sensor data and actual sensor data, a measure of similarity between the predicted sensor data and the actual sensor data, or measure of dependence between the predicted sensor data and the actual sensor data.

As an example, with respect to FIG. 2, the comparison module 250 may generate the uncertainty score 150 for a given time after receiving the sensor data 130b and the predicted sensor data 140, based on the sensor data 130b and the predicated sensor data 140. The uncertainty score 150 may be calculated by the microprocessor calculating the Euclidean distance between the sensor data 130b and the predicted sensor data 140.

The process 600 includes determining a control action for the device based on the measure of uncertainty (608). The control actions determined to be performed may be based on the measure of uncertainty. For example, each of the potential control actions may correspond to a measure of uncertainty threshold or a threshold range.

As an example, with respect to FIG. 1, the control actions for the powered exosuit 110 may include, for example, include one or more of reducing assistive force provided by the actuator(s) 120 of the powered exosuit 110 (e.g., reducing assistive force from 70% of maximum force to 40% of maximum force), stopping the assistive force provided by the actuator(s) 120 of the powered exosuit 110 (e.g., reducing assistive force from 70% of maximum force to 0% of maximum force), modifying the maximum flex angle of the powered exosuit 110 at the knee joint (e.g., reducing the maximum flex angle from 180° to 120°), modifying the minimum flex angle of the powered exosuit 110 at the knee joint (e.g., increasing the minimum flex angle from 20° to 60°), modifying the flex angle range of the powered exosuit 110 at the knee joint (e.g., modifying the flex angle range from 20°-180° to 60°-110°), activating other safety rules (e.g., enabling a predetermined safety rules meant to safeguard the wearer 102 such as, for example, increasing assistive force output, providing improved stability, reducing the allowable rotation at a joint, locking a joint to prevent rotation, or the like), modifying thresholds for performing an action (e.g., modifying the threshold corresponding to triggering a control action, such as reducing assistive force, to reduce uncertainty score required to trigger the control action, thereby increasing the likelihood that the control action will be performed at a future point in time), modifying an algorithm for determining a control value (e.g., adjusting a constant in an algorithm used determine the maximum flex angle when the control action of modifying the maximum flex angle or modifying the flex angle range is selected by the control system to be performed), and/or selecting a control program (e.g., selecting a safety program for the powered exosuit 110 in order to improve the stability provided to the wearer 102 in an effort to reduce the risk of injury). A control value may refer to a control setting of the powered exosuit 110, such as, for example, a joint angle or a percentage of maximum torque. The control system may select from control actions that are in addition to or in place of those control actions shown.

A number of implementations have been described. Nevertheless, it will be understood that various modifications may be made without departing from the spirit and scope of the disclosure. For example, various forms of the flows shown above may be used, with steps re-ordered, added, or removed.

A module (also known as a program, software, software application, script, or code) may be written in any form of programming language, including compiled or interpreted languages, and it may be deployed in any form, including as a stand-alone program or as a module, component, subroutine, or other unit suitable for use in a computing environment. A module does not necessarily correspond to a file in a file system. A module may be stored in a portion of a file that holds other programs or data (e.g., one or more scripts stored in a markup language document), in a single file dedicated to the program in question, or in multiple coordinated files (e.g., files that store one or more modules, sub programs, or portions of code). A module may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a module include, by way of example, both general and special purpose microprocessors, and one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

Embodiments of the invention may be implemented in a computing system that includes a back end component, e.g., as a data server, or that includes a middleware component, e.g., an application server, or that includes a front end component, e.g., a client computer having a graphical user interface or a Web browser through which a user may interact with an implementation of the invention, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products.

Thus, particular embodiments of the invention have been described. Other embodiments are within the scope of the following claims. For example, the actions recited in the claims may be performed in a different order and still achieve desirable results.

What is claimed is:

1. A method performed by one or more processors, comprising:
generating, by the one or more processors, forecasted sensor data representing an estimate of sensor data that will be produced by sensors of an exosuit at a particular time in the future, the exosuit being configured to assist mobility of a wearer;
obtaining, by the one or more processors, actual sensor data that is generated using the sensors of the exosuit, the actual sensor data representing a state or condition of the exosuit at the particular time;
determining, by the one or more processors, a measure of uncertainty based on the forecasted sensor data and the actual sensor data; and
based on the measure of uncertainty, determining, by the one or more processors, a control action for the exosuit to adjust assistance provided to the wearer.

2. The method of claim 1, wherein generating the forecasted sensor data comprises generating the forecasted sensor data representing an estimate of sensor data that will be produced by sensors of the exosuit at the particular time in the future.

3. The method of claim 2, wherein generating the forecasted sensor data representing an estimate of the sensor data that will be produced by the sensors of the exosuit comprises generating at least one of the following:
a forecasted joint angle of an angle sensor of the exosuit;
a forecasted force of a force sensor of the exosuit;
a forecasted pressure of a pressure sensor of the exosuit; or
an acceleration of an accelerometer or an inertial measurement unit of the exosuit.

4. The method of claim 2, wherein:
generating the forecasted sensor data representing an estimate of the sensor data that will be produced by the sensors of the exosuit comprises generating forecasted sensor data repeatedly for each of multiple successive time periods; and
determining a measure of uncertainty based on the forecasted sensor data and the actual sensor data comprises determining multiple measures of uncertainty each corresponding to one of the successive time periods.

5. The method of claim 4, wherein generating forecasted sensor data repeatedly comprises:
generating forecasted sensor data periodically; or
generating forecasted sensor data in response to detecting an event.

6. The method of claim 1, wherein the sensors comprise one or more of:
a force sensor;
a pressure sensor;
an accelerometer;
a position sensor;
an angle sensor;
a photoelectric sensor;
a time-of-flight sensor; and
an inertial measurement unit.

7. The method of claim 1, wherein determining the measure of uncertainty comprises determining a measure of similarity between the forecasted sensor data and the actual sensor data.

8. The method of claim 7, wherein determining the measure of similarity between the forecasted sensor data and the actual sensor data comprises comparing the forecasted sensor data with the actual sensor data.

9. The method of claim 1, wherein determining the measure of uncertainty based on the forecasted sensor data and the actual sensor data comprises determining a distance metric indicating a level of difference between the forecasted sensor data and the actual sensor data.

10. The method of claim 1, wherein determining the measure of uncertainty based on the forecasted sensor data and the actual sensor data comprises determining a vector distance between a first vector corresponding to the forecasted sensor data and a second vector corresponding to the actual sensor data,
wherein the first vector and the second vector each include values corresponding to multiple different sensors.

11. The method of claim 1, wherein determining the measure of uncertainty based on the forecasted sensor data and the actual sensor data comprises:
providing the forecasted sensor data and the actual sensor data to a machine learning model; and
obtaining the measure of uncertainty as an output of the machine learning model.

12. The method of claim 1, wherein determining the measure of uncertainty based on the forecasted sensor data and the actual sensor data comprises determining the measure of uncertainty based on one or more of:
multiple forecasts of sensor data corresponding to different time periods; or
multiple measurements of sensor data corresponding to different time periods.

13. The method of claim 1, wherein determining the measure of uncertainty based on the forecasted sensor data and the actual sensor data comprises determining the measure of uncertainty based on a comparison of outputs of multiple sensors of the sensors.

14. The method of claim 1, wherein determining the control action for the exosuit based on the measure of uncertainty comprises determining, based on the measure of uncertainty, to perform at least one of:
modifying control settings of the exosuit;
modifying a threshold for taking a particular control action;
activating one or more safety rules; or
selecting a control program to run.

15. The method of claim 14, wherein determining the control action comprises modifying the control settings for the exosuit, and wherein determining to modify a control setting of the exosuit comprises determining to modify at least one of:
a level of power assistance provided by the exosuit or a portion of the exosuit;
a maximum flex angle of a joint of the exosuit;
a minimum flex angle of a joint of the exosuit;
a joint angle of a joint of the exosuit; or
a maximum flex angle speed at a joint of the exosuit.

16. The method of claim 14, wherein determining the control action for the exosuit based on the measure of uncertainty comprises:
comparing the measure of uncertainty to a threshold measure of uncertainty; and
selecting a control action to perform based on the measure of uncertainty meeting the threshold measure of uncertainty.

17. The method of claim 1, comprising:
generating instructions to perform the determined control action; and
sending the instructions to one or more control devices.

18. The method of claim 17, wherein sending the instruction to the one or more control devices comprises sending the instructions to one or more of the following:
an actuator configured to provide rotational force at a joint of the exosuit;
an actuator configured to provide linear force between two or more components of the exosuit; or
a locking mechanism a joint of the exosuit.

19. A system comprising:
one or more computers; and
one or more computer-readable media storing instructions that, when executed, cause the one or more computers to perform operations comprising:
generating, by the one or more computers, forecasted sensor data representing an estimate of sensor data that will be produced by sensors of an exosuit at a particular time in the future, the exosuit being configured to assist mobility of a wearer;
obtaining, by the one or more computers, actual sensor data that is generated using the sensors of the exosuit, the actual sensor data representing a state or condition of the exosuit at the particular time;
determining, by the one or more computers, a measure of uncertainty based on the forecasted sensor data and the actual sensor data; and
based on the measure of uncertainty, determining, by the one or more computers, a control action for the exosuit to adjust an assistance provided to the wearer.

20. One or more non-transitory computer-readable media storing instructions that, when executed by one or more computers, cause the one or more computers to perform operations comprising:
generating, by the one or more computers, forecasted sensor data representing an estimate of sensor data that will be produced by sensors of a exosuit at a particular time in the future, the exosuit being configured to assist mobility of a wearer;
obtaining, by the one or more computers, actual sensor data that is generated using the sensors of the exosuit, the actual sensor data representing a state or condition of the exosuit at the particular time;
determining, by the one or more computers, a measure of uncertainty based on the forecasted sensor data and the actual sensor data; and
based on the measure of uncertainty, determining, by the one or more computers, a control action for the exosuit to adjust assistance provided to the wearer.
